(19) United States
(12) Patent Application Publication
(10) Pub. No.: US 2023/0197227 A1
(43) Pub. Date: Jun. 22, 2023

(54) HEALTHCARE DELIVERY SYSTEM

(71) Applicant: Barbara Czerska, Winter Park, FL (US)

(72) Inventor: Barbara Czerska, Winter Park, FL (US)

(21) Appl. No.: 18/108,279

(22) Filed: Feb. 10, 2023

Related U.S. Application Data
(63) Continuation of application No. 16/794,129, filed on Feb. 18, 2020.
Application No. 16/794,129 is a continuation-in-part of application No. 15/281,456, filed on Sep. 30, 2016, now abandoned.
(60) Provisional application No. 62/238,992, filed on Oct. 8, 2015.

(51) Int. Cl. G16H 20/00; G16H 50/70; G16H 80/00; G16H 50/30; G16H 10/60

(52) U.S. Cl. CPC G16H 20/00; G16H 50/70; G16H 80/00; G16H 50/30; G16H 10/60

(57) ABSTRACT

A healthcare delivery system that embodies methods of empowering collaborative multidisciplinary healthcare and the clinics that support them for providing everyday care to patients with chronic conditions, through in part by eliminating the current referral system to multiple specialists and providing collaborative multidisciplinary healthcare. A healthcare delivery that upends the traditional model where primary care physician, who has an essentially blind first visit with a patient, tries to develop plan of coordinated care in a very fragmented manner by referring patient to multiple specialist as needed. The present invention takes an unconventional ordering of combined steps to deliver improved healthcare drastically different from the traditional model.

### CROSS-REFERENCE TO RELATED APPLICATION

This application claims the benefit of priority of U.S. provisional application No. 62/238,992, filed 8 Oct. 2015, the contents of which are herein incorporated by reference. This application claims the benefit of priority of U.S. non-provisional application Ser. No. 15/281,456, filed 30 Sep. 2016, and claims the benefit of non-provisional application Ser. No. 16/794,129, filed 18 Feb. 2020 as a Continuation thereof, the contents of both are herein incorporated by reference.

### BACKGROUND OF THE INVENTION

The present invention relates to healthcare delivery systems and, more particularly, a healthcare delivery system that embodies methods of empowering collaborative multidisciplinary healthcare for providing care to patients with chronic conditions.

Healthcare spending in the U.S. is about 90% higher per capita than in most other industrial countries. The rising cost of health care is unsustainable. In 2010, healthcare costs consumed approximately 25% of budgets in the education sector and is expected to increase to over 40% by 2040 (Bill Gates TED talk on education). Health care delivery reforms, such as the Affordable Care Act, actually increased federal Medicaid spending by 18.4% in 2014. There is a need for cost effective comprehensive multidisciplinary care for the growing Medicaid population.

Over the last decade several reports showed that primary care physicians (PCPs) increased the number of referrals to specialist by 94%. Thus, specialists in the U.S. health care system play a particularly prominent role. Patient Centered Medical Home (PCMH) is a care delivery model whereby patient treatment is coordinated through their primary care physician to ensure they receive the necessary care when and where they need it, in a manner they can understand. PCMH thus inherently recognizes specialists as the people with whom they most frequently co-manage patients, or at least share them, and the people with whom they have (or should have) the most regular interaction. According to a recent study, however, only 62% of PCP's report getting consultation results from specialists, even though 81% of specialists report sending this information back to the referring PCP (O'Malley and Reschovsky, 2011). This suggests some substantial communication problems within the subsystem navigated by PCMH. A lack of clarity on the respective roles of PCP's and specialists undoubtedly may contribute to coordination issues among specialists and PCP's. Thus, significant fragmentation of care exists for patients with any chronic conditions, especially for patients with multiple chronic conditions.

In its 2001 report, the Institute of Medicine's Committee on the Quality of Health Care in America concluded that “between the health care we have and the care we could have lies not just a gap, but a chasm.” The Institute report underlined the ineffectiveness of our healthcare system's delivery model which is designed to treat the acute, sudden onset of symptoms in patients with little regard to treating those with chronic conditions. Moreover, shrinking access to specialists within each insurance network leaves significant gaps and creates barriers to care delivery even with the best primary care coordination. The shortage of primary care physicians makes it more difficult for special populations, like full-time employees or patients with multiple chronic conditions, to access primary, secondary or tertiary services.

Primary prevention aims to prevent disease or injury before it ever occurs. This is done by preventing exposures to hazards that cause disease or injury, altering unhealthy or unsafe behaviors that can lead to disease or injury, and increasing resistance to disease or injury should exposure occur. Examples include: legislation and enforcement to ban or control the use of hazardous products (e.g. asbestos) or to mandate safe and healthy practices (e.g. use of seatbelts and bike helmets); education about healthy and safe habits (e.g. eating well, exercising regularly, not smoking); and immunization against infectious diseases.

Secondary prevention aims to reduce the impact of a disease or injury that has already occurred. This is done by detecting and treating disease or injury as soon as possible to halt or slow its progress, encouraging personal strategies to prevent re-injury or recurrence, and implementing programs to return people to their original health and function to prevent long-term problems. Examples include: regular exams and screening tests to detect disease in its earliest stages (e.g. mammograms to detect breast cancer); daily, low-dose aspirins and/or diet and exercise programs to prevent further heart attacks or strokes; and suitably modified work so injured or ill workers can return safely to their jobs.

Tertiary prevention aims to soften the impact of an ongoing illness or injury that has lasting effects. This is done by helping people manage long-term, often-complex health problems and injuries (e.g. chronic diseases, permanent impairments) in order to improve as much as possible their ability to function, their quality of life and their life expectancy. Examples include: cardiac or stroke rehabilitation programs, chronic disease management programs (e.g. for diabetes, arthritis, depression, etc.); support groups that allow members to share strategies for living well; and vocational rehabilitation programs to retrain workers for new jobs when they have recovered as much as possible.

The transformation of primary care models first emerged in 2010 as a response to a need for change in the healthcare payments model. Thus, in primary care we observed a transformation of primary care practices and multi-specialist practices to a concierge medicine model. This model improved patient access to care and improved physician job satisfaction, however it did not change the role of the PCP and relationship in care delivery between the PCP and specialist. In concierge medicine we still see PCP referrals and care coordination even in the same multidisciplinary practices. The PCP has to refer a patient to a specialist in order to collect reimbursement for services from different payers and each specialist visits are scheduled separately.

The traditional fee-for-service system lacks efficiency and causes a lot of frustration not only to patients but also to practicing physicians. Building a new primary-care-for-value based payment model uses two strategies: first, building a new model from the ground up and disrupting the existing system, and second, applying innovations within the system and bringing that reconfiguration to traditionally structured practices.

Further compounding the problem, the shortage of primary care physicians makes it more difficult for special populations, like full-time employees or patients with multiple chronic conditions, to access primary or secondary preventive services. Current projections suggest that by 2020, there will probably be an additional 15 million Americans with hypertension, 12 million with diabetes, 4 million with coronary heart disease, 2 million with stroke, and 2 million with heart failure.

There is a growing need for better healthcare delivery for patients with chronic conditions, who more often need and end up using specialist care, and who will represent over 50% of the US population by 2020 (3). Despite spending the most in the world on healthcare there has been an increase in preventable existing chronic conditions over the last two decades. A review of chronic conditions by G. Anderson showed that patients with chronic conditions see at least three to five specialists and they utilize healthcare more often. The cost of care for patients with chronic conditions is higher than the general population with differences in quality of care stemming from gender and racial differences. Patients with chronic conditions spend more for healthcare and experience more hardship with paying medical bills while physicians taking care of patients with chronic conditions believe that the needs of these patients are not being met and physicians report difficulties in coordinating care of patients with chronic conditions. This is not surprising, since the way physicians are trained and the way the system is set up is to quickly put out small fires, such as a broken leg, while a chronic condition may require a lifetime's worth of care, guidance, and oversight on the physician's part, as is the case with heart conditions and diabetes, for instance.

Although specialist care is cited as being at least 10 times more expensive in the US than primary care, the only attempts at decreasing such costs have been through employing a “gate keeping” strategy to primary care, which ultimately results in more inefficiency and greater distrust among patients.

In 2012, studies showed that patients tend to prefer to see specialists over a general practitioner. In fact, it has been shown that the majority of all ambulatory visits, 55%, were in a specialist's office. Patient preference of care delivery by specialists can be measured by the number of self-referred new visits to specialists. Thus, among the Medicare population 1 in 5 new visits to specialist are self-referred, and 1 in 4 new visits among private insurance beneficiaries are self-referred despite frequently higher required copays for a given visit.

The currently existing primary care delivery models adopt the Center for Medicare & Medicaid Services (CMS) developed concept of PCMH. The driving force in the PCMH model is coordination, wherein a primary care physician delivers preventive primary care to a patient, and the same primary care physician coordinates advanced care through a web of specialists. Thus, significant fragmentation of care delivery exists for patients with any chronic conditions, and patients with multiple chronic conditions, who represent 50-70% of the general population, are forced to see three to four different doctors each at different offices. Despite spending the most in the world on healthcare there has been an increase in preventable existing chronic conditions over the last two decades.

In short, when primary care physicians coordinate advanced care and send referrals to specialists to secure secondary preventive services the already fragmented healthcare delivery process is made even more inefficient and fragmented for patients, especially those with chronic conditions.

As can be seen, there is a need for a healthcare delivery system that embodies methods of empowering collaborative multidisciplinary healthcare for providing care to patients with chronic conditions, thereby delivering preventative medical care that is of higher than average quality, much less expensive, and much safer. This kind of care will eliminate fragmentation, improve access, decrease variance in care delivery, and allow patients easier participation in their own health.

Furthermore, US National Center for Health Statistics describes chronic diseases as illnesses that generally cannot be prevented by vaccines or cured by medication, nor do they just disappear. About 15 percent of Americans between ages 20 to 44 years, 35 percent of Americans aged 65 to 79 years have chronic conditions. Approximately two-thirds of Medicare beneficiaries over age 65 have two or more chronic conditions, and approximately one-third have four or more. The current model of care delivery for these very complex patients is based on patient-centered primary care services, where a primary care physician (PCP) act as the coordinator of care and refers patients with chronic conditions as needed to a specialist or frequently to needed multiple specialist, as illustrated in FIG. 2.

Many barriers to care delivery are inherent in this model and include, for example, limited access to specialist, lack of coordination between multiple specialist and increased risk for medical errors due to multiple plans of care presented to the patients by each specialist and PCP.

The diagnosis of chronic conditions, especially multiple chronic conditions, carries very significant increase in cost of care delivery. This staggering cost of care triggered development of tools such as scoring systems that allows large care providers, health care systems and insurance companies to predict and estimate cost of care for high-risk patients with multiple chronic conditions (Lee, prior art). These scores are not used in the primary care physician offices since they cannot be applied to direct care delivery, for instance, they cannot be applied to instruct which medication should be used to treat the symptoms and prevent worsening of diseases. The scores are used by executive administrative teams to calculate cost effectiveness of care delivery and distribute resources to the sectors or practices that can provide maximal savings for the system.

The present invention proposes to develop new type of outpatient clinic that can deliver every day and advanced care to a patient with chronic conditions, as illustrated in FIGS. 3 and 5. The present invention takes an unconventional approach and combines a unique arrangement of well-defined steps in medical care delivery with the novel applications to deliver improved healthcare for patients with chronic conditions that drastically different from the traditional model.

A paradigm of this modern medical approach is that the patient's body can be seen by multiple specialists as a representation of a “meta-organism,” a complex, intercommunicating system of trillions of bacterial cells that coexist with a roughly equal number of human cells in the body.

Key paradigmatic terms include:

Specialist: a physician with additional clinical training, fellowship added after primary care training in the field of expertise. Board certified as specialist definition does not include primary care certification of family medicine certification for the purpose if this invention.

Microbiota: This term refers to a collection of all taxa constituting microbial communities, such as bacteria, archaea, fungi and protists. When it refers to a specific environment, the term is preceded by the said location, for example, ‘the gut microbiota’ refers to the intestinal tract and the ‘oral microbiota’ is used when speaking about all the microbes from the oral cavity.

Microbiome: This term was initially used to refer to the genes harbored by microbes; however, currently, the term ‘microbiome’ is also commonly used to refer to the microorganisms themselves (i.e., the microbiota).

Probiotics: These are live microorganisms that, when administered in adequate amounts, confer a health benefit to the host.

Prebiotics: These are substrates that are selectively utilized by host microorganisms conferring health benefits. The probiotics given together with prebiotics are called symbiotics.

The ordered combination of various ad hoc and automated tasks in the presently disclosed method of care delivery necessarily achieves improvements through the specifically arranged processes described in detail herein. In addition, the unconventional and unique aspects of these specific processes represent a sharp contrast to merely providing a well-known or routine environment for performing a manual or mental task.

### SUMMARY OF THE INVENTION

In one aspect of the present invention, disruption is delivered to the presently existing standards of everyday care delivery for complex patients, like those with chronic conditions, includes development of novel clinics for patients with chronic conditions. The disruptive aspect includes therefore a change in the standards of process of care delivery for patients with chronic conditions that is achieved by assigning to each clinic a plurality of physicians specialists based on synergistic knowledge of each specialist in the multiorgan pathophysiological processes occurring in chronic conditions, the plurality of physicians specialist comprising an immunologist, an infectious disease specialist, a cardiologist an endocrinologist and a gastroenterologist, implementing a collaborative model of practice between specialist, providing a method of delivering healthcare services to patients with chronic conditions through collaborative multispecialty team in the clinic that can utilize an online database; electronically storing in on line database with a signature medical history describing a patient having at least one chronic condition providing an online database through the following: a method of delivering healthcare services to patients with chronic conditions through collaborative multidisciplinary healthcare including the following: providing an online database; electronically storing in the online database a signature medical history describing a patient having at least one chronic condition; calculating a risk score related to primary, secondary, and tertiary care associated with the at least one chronic condition based in part on the signature medical record; formulating a plan of care for the patient based on the signature medical record and on the plurality of physician specialists, wherein the plan of care is formulated before a first visit; and providing a collaborative consultation between the patient and each of the plurality of physician specialists within a predetermined time frame of the first visit, thereby eliminating a primary physician referral of the plurality of physician specialists, wherein the predetermined time frame begins and ends at the first visit, wherein the risk score is based on a set of immune modulations in each patient, wherein the set of immune modulations includes a presence of “fingerprint” of gut microbiome, wherein the plan of care includes one or more immune modulating therapies, wherein the plan of care is based in part on a presence of “fingerprint” of gut microbiome through a comprehensive stool study, wherein the plan of care is based in part on a presence of “fingerprint” of gut microbiome through a determination of the following: blood levels of inflammatory markers, erythrocyte sedimentation rate, acute phase proteins and immune activation markers and stool studies.

In another aspect of the present invention, such method includes providing an online database that includes not only population health data but adds the unique “fingerprint” of each patient immune, inflammatory state and gut microbiome; electronically storing in the online database a signature medical history describing a patient having at least one chronic condition; calculating a risk score related to primary, secondary, and tertiary preventive care associated with the at least one chronic condition based in part on the signature medical record; assigning a plurality of physician specialists based in part on the signature medical record, the risk score, and the population health data; formulating a plan of care for the patient based on the signature medical record and on the plurality of physician specialists, wherein the plan of care is formulated before a first visit; providing collaborative consultations between the patient and each of the plurality of physician specialists during the first visit, thereby eliminating a primary physician referral of the plurality of physician specialists and changing a standards of “patient flow” process in primary are; compiling and electronically storing a file of the first visit on the online database; predicting risk of medical complications after the first visit based in part on the plurality of physician specialists and the signature medical record; establishing at least one procedure to address the predicted risk of medical complications, whereby decision-making times are decreased upon onset of one of the predicted medical complications; documenting and electronically storing the plan of care in the online database; instructing the patient on the following: following an individual health plan based on the plan of care; and accessing the signature medical history and said file of the first visit through the online database; providing the patient with at least one mobile electronic device for tracking progress of the individual health plan, wherein the at least one mobile electronic device stores output on the online database; and contacting the patient in person or through virtual communications for tracking compliance of the individual health plan.

In yet another aspect of the present invention, innovations are implemented within healthcare delivery that combines modern technology and its convenience with collaborative effective multi-specialty care, wherein such care is value based and run through a novel clinic includes establishing a clinic for delivering healthcare services to patients with chronic conditions, including matching a plurality of physician specialists to a predetermined population by determining a plurality of most frequently occurring types of chronic condition based in part on a population health information disclosure of the predetermined population; providing basic laboratory equipment; determining procedures and tests to be performed by each physician of the plurality of physician specialists based in part on a limited scope preventive services model, wherein the limited scope preventive services model includes only non-invasive testing and procedures within a relevant specialty; and eliminating a primary physician referral of the plurality of physician specialists.

In yet another aspect of the present invention, the novel clinic method further includes providing an online a novel database that includes not only a population health data but adds a unique “fingerprint” of each patient immune and inflammatory state and gut microbiome; electronically storing in the online database a signature medical history describing a patient having at least one chronic conditions; calculating a risk score related to primary, secondary, and tertiary preventive care associated with the at least one chronic condition based in part on the signature medical record; assigning at least one quarterback physician specialist of the plurality of physician specialists to coordinate care among the plurality of physician specialists based in part on the signature medical record and risk score; formulating a plan of care for the patient based on the signature medical record and on the at least one quarterback physician specialist, wherein the plan of care is formulated before a first visit; providing collaborative consultations between the patient and each of the at least one physician specialist during the first visit; compiling and electronically storing a file of the first visit on the online database; predicting risk of medical complications after the first visit based in part on the at least one physician specialist and the signature medical record; establishing at least one procedure to address the predicted known risk of medical complications for given conditions and record all new data to detect unknown correlations between microbiome and clinical symptoms or diagnosis, whereby decision-making times are decreased upon onset of one of the predicted medical complications; documenting and electronically storing the plan of care in the online database; instructing the patient on the following: following an individual health plan based on the plan of care; and accessing the signature medical history and said file of the first visit through the online database; providing the patient with at least one mobile electronic device for tracking progress of the individual health plan, wherein the at least one mobile electronic device stores output on the online database; and contacting the patient in person or through virtual communications for tracking compliance of the individual health plan.

In yet another aspect of the present invention, the method of improving delivering healthcare services to patients with chronic conditions through a pre-existing and newly developed clinic that implements a new model of collaborative multidisciplinary healthcare, includes the following: the plurality of physician specialists determined as an immunologist, an infectious disease specialist, a cardiologist, an endocrinologist and a gastroenterologist; including a novel approach to standard laboratory testing for patient with chronic condition and standard equipment provided in clinic that allows comprehensive functional evaluation of individual patient immune, and inflammatory responses in patients. Our testing equipment was chosen to correlate with the fields of expertise of practicing physicians. The present invention includes introduction of new paradigm to primary care—everyday care that the patient's body is seen by multiple specialist as a representation of a “meta-organism,” a complex, intercommunicating system of trillions of bacterial cells that coexist with a roughly equal number of human cells in the body. The present invention includes a signature medical history describing a patient from said pre-existing clinic and combining it with novel immune, inflammatory and microbiome personalized data. The present invention includes calculating a risk score related to primary, secondary, and tertiary preventive care associated with the at least one chronic condition based in part on the signature medical record; the scoring system of the present invention may be used not only for prediction of one acute event but for evaluating of impact of interventions and treatments. The score will serve to evaluate the need of immune modulating therapies and monitoring outcomes. The present invention includes assigning a plurality of physician specialists based in part on the signature medical record and the risk score. The present invention includes formulating a plan of care for the patient based on the signature medical record and on the plurality of physician specialists, wherein the plan of care is formulated before a first visit; and might include new immune modulatory therapies. The present invention includes assigning a quarterback physician specialist to coordinate multispecialty plurality of physicians during single visit and execution of plan of care based in part on signature medical records. The present invention includes providing a collaborative consultation between the patient and each of the plurality of physician specialists within a predetermined time frame of the first visit, thereby eliminating a primary physician referral of the plurality of physician specialists, providing an online database, and electronically storing in the online database, a signature medical history describing a patient having at least one chronic condition.

In another aspect of the present invention, a treatment method includes the following: determining a fingerprint of gut microbiome measured in a biological sample from a patient diagnosed with a chronic condition to identify the patient having at least one immunological marker and at least one inflammatory marker; administering a treatment to the patient having an immunological marker and an inflammatory marker of the chronic condition, wherein the treatment is a course of immune modulation therapy capable of treating the chronic condition, wherein the course of immune modulation therapy comprises administering prebiotics, probiotics, symbiotics, antibiotics or biological stimulators, wherein the course of immune modulation therapy comprises an assessment from an infectious disease specialist based on the at least one immunological marker in view of the fingerprint of gut microbiome.

In yet another aspect of the present invention, the method further includes wherein the course of immune modulation therapy comprises an assessment from a cardiologist based on the at least one inflammatory markers of the patient in view of the fingerprint of gut microbiome, wherein the course of immune modulation therapy comprises an assessment from a gastroenterologist based on an immune status of the patient in view of the fingerprint of gut microbiome, wherein the course of immune modulation therapy comprises an assessment from a gastroenterologist based on a gastrointestinal tract of the patient in view of the fingerprint of gut microbiome, wherein the course of immune modulation therapy comprises an assessment from an endocrinologist based on or more pathophysiological markers of the patient in view of the fingerprint of gut microbiome, wherein said assessments consider a signature medical history of the patient; further including calculating a risk score related to primary, secondary, and tertiary preventive care associated with the patient based in part on the signature medical record; further including formulating a plan of care for the patient based on the signature medical record and on a plurality of said specialists, wherein the plan of care is formulated before a first visit with the patient; and further including providing a collaborative consultation between the patient and each of the plurality of said specialists within a predetermined time frame of the first visit, thereby eliminating a primary physician referral of the plurality of said specialists, wherein the fingerprint of gut microbiome is the genetic signature of gut microbiota' refers to the gastrointestinal tract of the patient, wherein the fingerprint of gut microbiome is the determined through stool studies of the patient, and wherein the plurality of assessments is based in part on a blood levels of inflammatory markers, an erythrocyte sedimentation rate, and acute phase proteins.

These and other features, aspects and advantages of the present invention will become better understood with reference to the following drawings, description and claims.

### BRIEF DESCRIPTION OF THE DRAWINGS

FIG. 1 is a schematic representation of an exemplary embodiment of the present invention, illustrating expanded levels of services through the proposed novel clinics;

FIG. 2A is a schematic representation of an exemplary embodiment of the present invention, illustrating patient flow process through the proposed novel clinics;

FIG. 2B is a flow chart of the exemplary embodiment illustrated in FIG. 2A of the present invention;

FIG. 3A is a schematic representation of an exemplary embodiment of the present invention, demonstrating a collaborative-synergistic care model and patient flow process;

FIG. 3B is a flow chart of the exemplary embodiment illustrated in FIG. 2A of the present invention;

FIG. 4 is a schematic representation of an exemplary embodiment of the present invention; and

FIG. 5 is a schematic representation of an exemplary embodiment of the present invention, showing connectivity with the multidisciplinary teams, and importance of data collection and analysis.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a healthcare delivery system that embodies methods of empowering collaborative multidisciplinary healthcare for providing care to patients with chronic conditions.

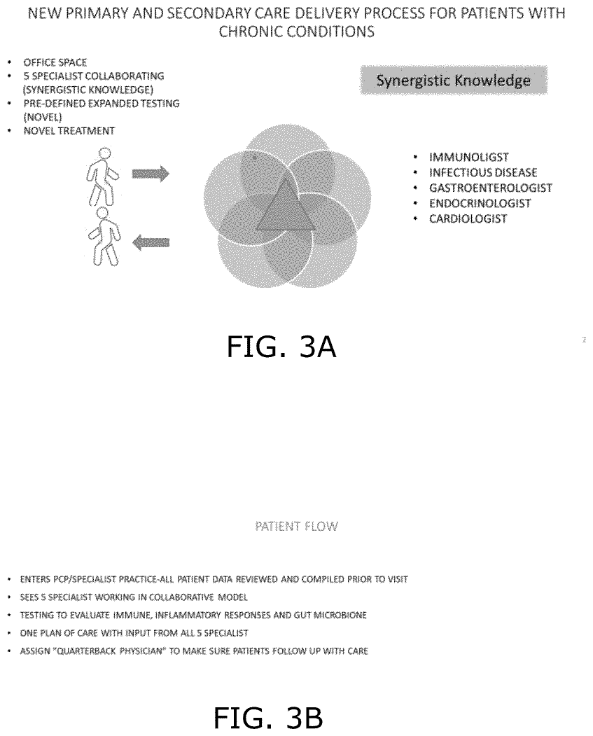

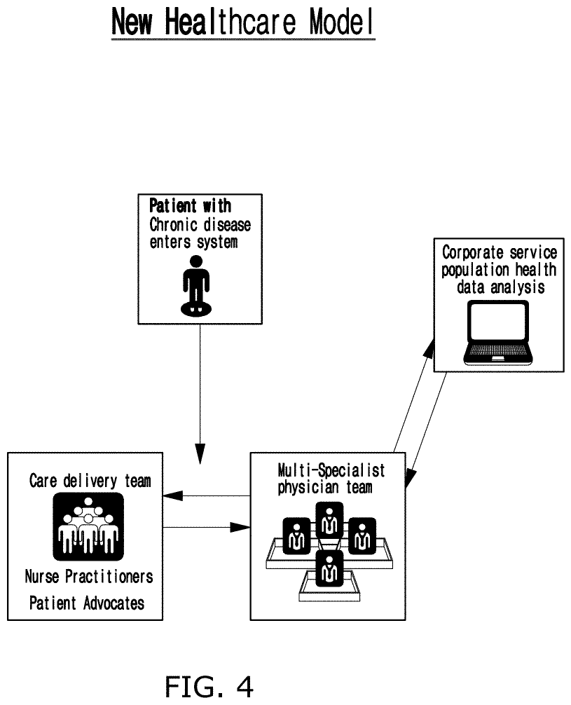

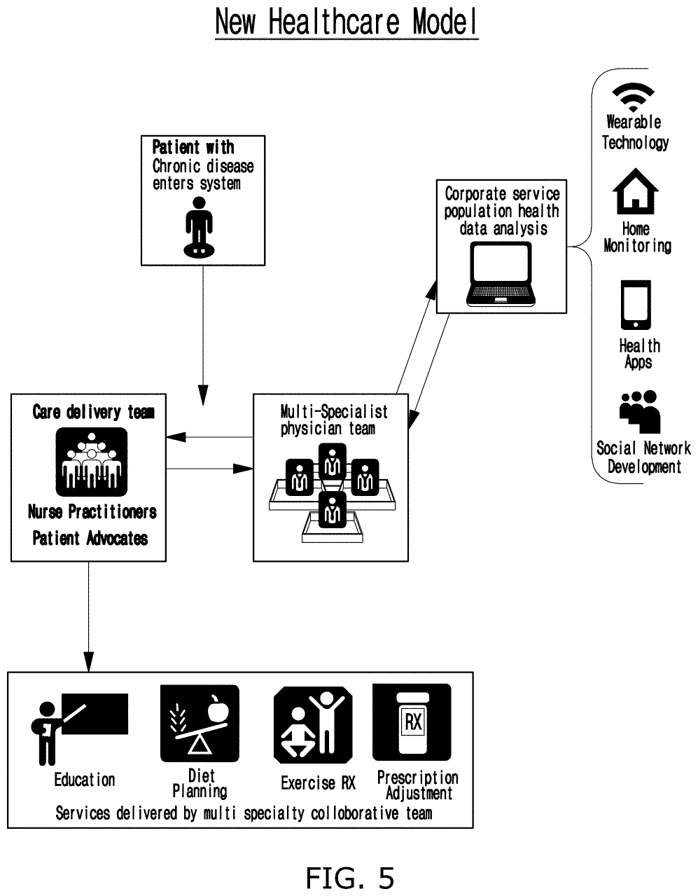

Referring to FIGS. 3 through 5, the present invention may include use of at least one computer with a user interface. The computer may include at least one processing unit coupled to a form of memory. The computer may be any computing device including, but not limited to, desktops, laptops, and smart devices such as a tablet, smart watch, smart phone, and the like. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The present invention may include a healthcare delivery system that relies on the power of collaborative multidisciplinary expert care, enhanced by standardized care, and empowered by modern technology for providing practices processes, protocols and guidelines. The present invention delivers a wider spectrum of services in everyday primary care, eliminates time consuming need for referrals to specialists, eliminates cost associated with referrals like co-pays, gives patient easier access to care and allows wider implementation of primary, secondary and tertiary preventive care.

The invention teaches away from the existing industry standard of primary care delivery (even for patients with chronic conditions) by single generalists empowered by healthcare extenders whose roles are defined as care coordinators. The present invention introduces noninvasive specialists with clearly defined roles to deliver collaborative, comprehensive primary and secondary and tertiary care. The present invention delivers a wider spectrum of services in everyday primary care, eliminates the time consuming need for referrals to specialists, eliminates cost associated with referrals like co-pays and unnecessary testing, gives patient easier access to care, and allows wider implementation of primary, secondary, and tertiary noninvasive and preventive care.

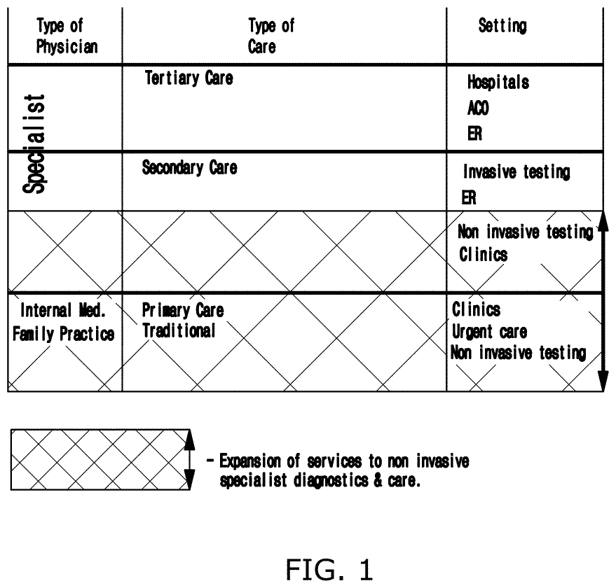

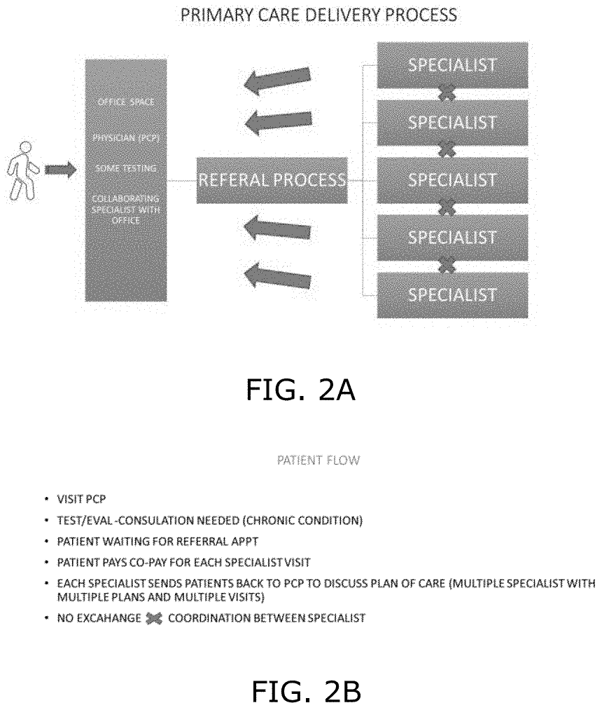

The present invention may eliminate the primary care physician's role of coordination and referrals and replaces it with a novel multidisciplinary collaborative specialist approach to care that can be delivered in the same office practice, as illustrated in FIG. 3. The collaborative multidisciplinary specialist care will deliver primary, secondary, and tertiary preventive services within the scope of practice described by the CMS definition of primary care services, but can expand the scope of services to non-invasive specialist care, as illustrated in FIG. 1.

### The Difference Between the Present Invention and Existing Practices and Published Models for Chronic Care Delivery Solutions

The present invention provides a novel approach and/or method to care delivery by replacing the role of a general practitioner as a care coordinator with the centrally located and collaborative care of a multidisciplinary and multispecialty team. The multispecialty expert team is assembled based not only on analytics of a given population's health under the team's care and takes into account not only traditional treatment of symptoms and complications of chronic diseases but looks into the pathophysiology of human responses in immune, inflammatory and gut microbiome of each individual. The synergistic knowledge of specialists allows to undercover correlations between multiple chronic conditions, their symptom, and complications. The multidisciplinary experts allow to understand better the pathophysiology of chronic conditions and expand treatment to new modalities. This approach allows: better access, higher quality care, increased safety, lower cost, and less fragmentation in patient care. A collaborative multidisciplinary approach decreases waste in care delivery caused by the duplication of services by placing the one most appropriate specialist in charge of each case. In the past, a patient would visit a general practitioner if he were feeling ill, the general practitioner would run tests to determine a diagnosis and if that physician was confused or concerned would then refer the patient to a specialist who would then run the similar or more tests to determine her own diagnosis. The patient would be charged for both visits and all the tests and would have to coordinate with at least two different physicians on various health matters. This increases cost, effort, and time dedicated to simply keeping track of which doctor's appointment is when and where.

The assignment of a specialist in charge (a “quarterback”) is done based on patient's clinical data and risk scores, one provider overseeing a more holistic approach to patient care. Currently, the lack of direct access to specialist services is typically substituted with emergency room visits. The model helps to save resources and time for patients with multiple chronic conditions by making available a multi-specialty consult visit in one day and in one location. The value of getting a unitary, comprehensive plan of care developed by a group of specialists all focusing together becomes a crucial entity for patient compliance, self-engagement in care, and for case management of chronic conditions. Simply stated, by eliminating a care coordinator who has to communicate with various different physicians and diagnosticians in any number of external offices (any of whom may or may not be in a patient's insurance network) we are eliminating a significant amount of cost, time, and effort for the patient, the physicians, and the medical insurance companies, commonly referred to as payers within the healthcare industry.

With modern electronic medical records (EMR's) the present invention allows for a review of each member of a given population's health with a unique scoring system for multiple risk factors. The present invention can easily analyze claims data from payers to obtain better understanding of total cost of care for each patient. Insurance claims data traditionally was not accessible and utilized by providers. By analyzing these factors, the Clinic can also estimate the potential cost of care for a given population using well recognized statistical models such as the well-established Dartmouth model. This approach allows for the implementation of target based primary and secondary prevention measures and wellness programs.

This model allows the delivery of health management programs for an entire population of patients through real time analytics of data and fast interventions at the first sign of any clinical trouble. The present invention will operate within a community similarly to a PCMH or a Specialist Centered Medical home and includes each of the six NCQA elements of care, which will allow it to easily connect, oversee, and follow any external health care “neighbors” and networks.

A clinic of record structure aligned with a novel value-based payment models allows for the creation of unique cost saving approaches. Thus, the revenue driver is not represented by utilization of clinical resources, such as diagnostic tests, follow-up visits, and referrals, but rather the number of members entering the solution.

The novel concept of care delivery has highly standardized processes and protocols which utilize evidence-based medicine and clinical decision making tools embedded into the daily flow of practice and powered by novel information technology and electronic medical records. Moreover, the present invention is configured to be adaptive to improvements in technology: for example, improvements with novel new solutions in health information technology like dynamic modeling of data; improvements with new point of care diagnostic tests; new patient self-monitoring devices; and patient and clinician engagement tools.

The present invention embodies methodologies regarding establishment of clinics; delivery of care through said clinics; and monitoring and adapting said clinics so that they function in accordance with the present invention as described herein.

### Method of Establishment of Clinics

Steps 1-17 may be adapted to put in place how a clinic using the invention will function. Working with the given population a clinic has predetermines\d assembled specialist physicians chosen based on synergistic knowledge of each specialist in multiorgan pathophysiological processes occurring in chronic conditions. It then decides which protocols and procedures to put in place to assure the best quality of healthcare delivery for the given patients. Some of the procedures are mandated by governmental jurisdictions, some are industry standards, while others are specific to the clinic or physicians.
1. Gather data for a population from an already existing community's health network and payers.
2. Merge clinical data and available payers claims data.
3. Study the population data.
5. Assemble the multi-specialty team of physicians, nurse practitioners and patient advocates.
6. Determine the scope of service protocols for each specialist and determine the procedures and protocols of collaborative care delivered by all specialist. Determine what procedures, tests, and services each physician will be able to perform based on the scope of preventive services within a given specialty. Only non-invasive testing or procedures should be performed due to the predetermined basic laboratory equipment with certified Clinical Laboratory Assistant (CLA) waived testing equipment located within a given clinic. Further factors include, but are not limited to, proximity to a conventional hospital and any additional equipment and facilities contained within a given clinic. Because a clinic's focus will be on preventive medicine and standard non-invasive care, urgent care or any kind of surgery or procedures requiring anesthesia should not be performed within a clinic location.
7. Apply the determinations of scope of service protocols for each specialist and detail them through an established system of Current Procedural Terminology codes (CPT). CPT codes are an already established coding system used by healthcare facilities to bill insurance companies and are standard in the healthcare industry. At the present time they are described as ICD 10 codes and/or CPT codes. All provided services within a clinic will be coded which can be later utilized for analysis of data and establishing the value of providing services as compared to traditional health system care delivery.
8. Assemble the needed space and CLA equipment that allows collaborative work among a clinical team.
9. Develop clinical policies and protocols for the standardization of work flow; the decrease in fragmentation; and the tolerable variance in care delivery, which allows a clinician to alter his decisions based on a patient's clinical status within a given range of treatment and/or health variance, using industry standards, best-practices data, professionally accepted guidelines, and government mandates.
10. Train all clinical staff in the novel culture of collaborative care, the clinical policies, and technologies to be used.
11. Customize commercially available Electronic Medical Records (EMR) for a given clinic's and population's needs and track evidence of multidisciplinary care for a given patient. For instance, if a given population predominantly has heart problems the EMR's would be required to track heart rate, blood lipids profiles, family history, blood pressure, and exercise but would not need to track bone density or other non-related factors.
12. Based on the principles of already established Evidence Based Medicine (EBM), develop Clinical Decision Support Tools (CDS tools), which are a set of rules and protocols, into EMR.
13. Develop pathways of care and the procedures related to each specialty and domains of health care, including but not limited to, protocol/procedure for patient intake, registration, visits, follow up, and active remote case management. Develop a treatment plan using industry standard, best-practices data, professionally accepted guidelines, government mandates, and the most currently available medical practices that will ensure a particular patient's well-being while minimizing time, effort, and cost to that patient. Also develop standardized procedures to help streamline decision-making on part of the physician for a given patient's treatment plan based on the total factors surrounding a patient's health including, but not limited to, pertinent medical test scores and feedback from the patient. These plans and protocols based on a patient's condition or diagnosis will allow for streamlining decisions of any additional needed testing or treatments and can be used by physicians and nurses.
14. Develop electronic templates for electronic records; first visit records; multidisciplinary team notes composed of all of the pertinent staff's impressions and recommendations; multidisciplinary collaborative consults which would occur in a single visit to the clinic, all of the pertinent physician specialists seeing a patient within a predetermined time frame to decrease the amount of time needed to see different specialists for varying conditions; annual and wellbeing records, virtual visit records, urgent visit records, case management, follow up of plan of care, follow up on patients' health goals, personalized teaching, integration of holistic medicine. The predetermined time frame being a function of the severity and multiplicity of the conditions to be treated, as well as the patient's signature medical record. For instance, if a patient has diabetes, coronary artery disease, and chronic asthma three specialists will see the patient at the same time and be able to put together a single, comprehensive health plan for the patient that will complement each other's goals instead of working against them; Making the collaborative notes from the multi-specialist team available to a patient during the same visit challenges the existing traditional standards of care, making it easier for a patient to follow his health plan or clear up any confusion should he have any questions.
15. Develop in EMR portal access for costumer's clinical data, teaching materials and clinic schedule.
16. Develop a portal for communications and transferring data to existing medical facilities like emergency rooms, hospitals, and pharmacies.
17. Compile all previous medical records released by patients and recorded answers to given questionnaires upon a patient's registration into a signature medical record.

The matching of a group of physician specialists to a population is critical to efficacy of the present invention. It is well recognized that majority of chronic conditions are classified now as an uncurable diseases. The pathophysiology of these conditions are not well explored, therefore applying the general rules of treatments similar for all patients without further inside to each individual response to diseases makes the treatment difficult and quite often unreliable. A large majority of the US population has at least one chronic medical condition that requires regular visits, examinations, and medications. By predicting which specialists a population will need to see most often, given a population's previously disclosed health information such as insurance claims or medical records and then analyzing that data to see the most frequently occurring types of illnesses (e.g. cardiovascular, immunological, etc.) costs can be lowered and quality of delivery can be increased by implementing customized, patient-centered care on a much smaller and individual basis. Thus, a clinic does not have to be built like a large hospital with a wide spectrum of costly services that might not be utilized in a specific population. This approach can help decrease the total cost of care delivery. Instead of seeing multiple physicians at multiple different offices at multiple different times a patient can see all of the physicians he would otherwise need to in a single place at one time. Time and monetary costs will decrease for a patient if he is able to see multiple physician specialists at a time. By contrast, in the current care delivery system since most specialists do not actively work or even coordinate together patient care can be scattered and confusing at best or work against each other at worst. By actively coordinating together an overall treatment plan can be formulated that is more effective, less costly, and ultimately much safer.

### Method of Delivery of Care Through Said Clinics

Steps 18-39 may be adapted to deal with how an individual patient will receive care. By using the invention along with current medical and IT technologies a higher quality of care along with a lower cost of delivery is assured. The patient is regularly contacted and monitored and his patient records recorded electronically in the EMR are updated regularly. All information is available to both him and the physicians in the clinic. Patients are then seen again by a physician, tests are done to see if he has objectively improved his health, and a new plan is implemented if needed and steps 32-39 are repeated as necessary.
18. Transfer all patient information into EMR database available to both the patient and physician before and after each visit.
19. Assign a head specialist quarterback in charge of each case based on patient clinical data in EMR.
20. Extract pertinent data from patient's signature medical record.
21. The extracted pertinent patient's data will be used by healthcare professionals for calculation of probabilities of risk of developing the most common complications associated with a specific diagnosis or for the evaluation of risks of developing common chronic conditions. For example, patients with high cholesterol and family history of heart attacks and high triglyceride and large waist size have a higher probability of having a heart attack than those who have low cholesterol and no family history of coronary artery disease. Those who have diabetes and have protein in urine have a higher probability of progressive renal complication of diabetes. Calculation of a pertinent risk scores (developed and used as standards in clinical guidelines) for each patient related to primary, secondary, and tertiary preventive care based on the clinical data from the patient's signature medical record will help to identify patients with highest risk and allow doctors to manage their condition more effectively in order to prevent potential complications or progression of existing condition. The patient's trend or trajectory in his objective health (e.g. if his test scores have been steadily getting worse) can be easily established based on scoring metrics. Additionally, there are several already existing, standardized disease-specific risk scores established for use in practice like heart failure scores, coronary artery disease scores, liver scores, and acute illness scores. The use of multiple risk scoring (utilization of combination of score for risk assessment) allows physicians to define a patient with special needs that may require more medical attention to prevent adverse health outcomes. Utilization of multiple specialist collaborative care in patient care with multiple high-risk scores can determine an effective plan of action that will take into account, all of his conditions from the beginning.
22. Code the risk scores and pertinent diagnosis electronically into a patient's signature record.
23. Formulate a plan of care based on existing history before first visit using the data from the patient's previously disclosed medical records combined with the latest in medical practice, industry standards, best-practices data, professionally accepted guidelines, and government mandates and any new test scores or observations from a physician that a patient visit within the clinic The current standards of care call for developing a plan of care for a patient only after the first visit, often not reviewing any pertinent medical data such as previous test scores or previous doctors' notes. Thus, usually within fifteen minutes of first seeing a patient a physician will formulate a plan from a very limited interview with a patient and that patient's self-reported diagnosis. The signature records developed in a clinic will include analyzed pertinent information from all previous clinical records and allow a physician to formulate long and short term plans of care before even seeing a patient and discuss those plans with the patient during that visit to see if he has any questions. The plans can later be augmented given a patient's future health status. This makes a patient's visit less stressful and will allow the patient to concentrate on recent history and current problems and gives a patient more time with a physician.
24. Based on clinical data and risk scores the head specialist decides and assigns additional specialist involvement if necessary.
25. Electronically deliver the requests for collaborative care to each specialist mail box through EMR.
26. Review of patient care plan in EMR before visit by patient advocate and nurse practitioner.
27. Schedule in house tests and diagnostic procedures if needed in addition to standard physical examinations.
28. During his visit the patient is examined and tests are conducted, the results of which are given to the patient during the actual visit while also uploaded to the EMR database.
29. Review the patient's previously developed personalized care plan with him, correcting it based on any new findings if needed.
30. Give the patient the treatment options, prescriptions, visit notes and tests results at the end of his visit.

In certain embodiments, improvements in technology (such as internet-connected medical devices, miniaturization of testing equipment, smartphones, tablets, home computers, new software) may enable the following steps:
31. Give the patient instructions on how to follow his individual health plan and how to access his medical records and educational materials in the central database online.
32. Give patients internet-connected devices that monitor their health status (such as weight, heart rate, blood pressure, or other pertinent factors) to track their own progress if needed, the data of which is automatically uploaded into the EMR database. Devices such as digital scales, blood pressure cuffs, and smartphone-based heart-rate monitoring can be connected with a patient portal, which allows a patient to review all of his medical information including staff notes, health care plan, and test scores.
33. Review the home recorded data now available to the physician in the EMR.
34. Contact the patient on a regular basis to track his progress in his care plan, the new information likewise uploaded into the EMR database.
35. Review the data, ensuring the entire clinical team assigned is involved.
36. Decide if intervention is necessary before the patient's next visit.
37. Have the patient visit either in person or through virtual communication means.
38. If new tests and exams are required give the patient his results during the follow up visit while also uploading them into the EMR database.
39. Give the patient a new individualized health plan, instructions on how to follow it, and a list of options for his care, at the end of his follow up visit.

The protocols of care and treatment plans as discussed in 13 and 23 that have been determined based on current medical knowledge and practice, industry standards, best-practices data, professionally accepted guidelines, and government mandates. The protocols of care are also important. Given the amount of patients, standardized procedures are necessary to ensure both efficacy of care and patient safety. Knowing that patients will have a specific chronic condition allows for a clinic to construct a standard protocol for treatment. Further, given that patients will most likely have a chronic condition, establishing predetermined specific procedures dealing with an array of different medical complications will decrease decision-making times for physicians, thus ensuring higher quality of care for patients; lower mortality and morbidity rates; and ultimately lower costs of care delivery.

### Monitoring and Adapting Said Clinics

Steps 40-47 may be adapted to demonstrate how a clinic using the invention will continue to improve by measuring its own data, analyzing it, and implementing methods on how to improve its outcomes.
40. Report standard quality and patient satisfaction data.
41. Develop own specific quality measures to be used in a quality improvement plan based on, among other things, effectiveness of care in multiple chronic conditions, cost savings to patients, outcomes of care delivery, access and utilization of telemedicine, and the mortality and morbidity rates of a specific population as compared to national standards in addition to industry and government standards.
42. Measure your own outcomes to determine if standards are met.
43. Implement the quality improvement plan.
44. Develop patient's registries.
45. Study and report each registry's outcome.
46. Develop data analyses of total cost of services.
47. Calculate the value of service and potential savings for each costumer.

Regularly monitoring and communicating with patients is necessary given the likelihood of chronic conditions. Efficacy of treatment and patient safety require the judgment and experience of a physician to make decisions based on incoming data. The model will deliver population health management programs with real time analysis of data and safety metrics allowing early fast interventions at the first sign of any clinical trouble.

By following the above listed steps a multidisciplinary specialist team of physicians will be able to deliver preventative health care to patients with chronic conditions which will improve the quality of individual care, lower the costs of delivery, and improve patient safety.

The order of the steps generally cannot be changed given the fact that the staff, training, space, and information technology elements of the invention need to be in place before a clinic starts to receive patients. However, the present invention can be used to transform an already existing clinic to follow the methods described herein to improve efficiency and patient safety. The present invention can also be constructed for use as a remote communication solution with the aid of teleconferencing software such as Skype and Facetime and hardware such as webcams and smartphones with virtual visits and live access to collaborative multidisciplinary specialist care with the availability of in-home advanced health IT and limited self-diagnostic point of care tests. The present invention can be deployed in remote and less populated areas especially with patients with multiple chronic conditions. The present invention can also utilize a multidisciplinary approach to chronic case management should a patient have more than one chronic condition. Additionally, insurance providers can provide the present invention as a solution option in their health plans to decrease their costs, thus reducing the amount of money their customers spend.

Referring to FIG. 3 through FIG. 5, the present invention may include the following. A method of improving delivery of healthcare services to patients with chronic conditions for a pre-existing and new primary care setting clinic, including the following systemic steps: assigning a plurality of physician specialist to a pre-existing clinic as a function of both prior insurance claims associated with the pre-existing clinic and laboratory equipment located at the clinic. The plurality of physician may include the following five (5) specialists: immunologist, infectious disease specialist, cardiologist, endocrinologist and gastroenterologist. Assigning or re-assigning the plurality of physician specialist and laboratory equipment to the clinics is a process that involves multiple levels of decision-making, wherein the prior insurance claims assist to define the presence of diseases and illnesses behind the relevant chronic conditions.

An advantageous step in determining the arrangement of the plurality of physician specialists to novel care model involves incorporation of recent discoveries in the area of human microbiome and its potential involvement in many chronic conditions. Specifically, new research points to involvement of existing microbes in the human gut in the vital human processes like metabolism and immune responses. It appears that these bacteria communicate in ways that are poorly understood and may have important effects in many conditions not traditionally considered infectious diseases. These include cardiovascular disease, diabetes, psychiatric and neurologic diseases, asthma, obesity, the metabolic syndrome, cancers, inflammatory bowel disease, and even therapeutic responses to medications and vaccines.

The plurality of physician specialist may develop complimentary and synergistic impact on diagnosis and treatment of multiple disorders and will allow with time to implement new treatments based on evaluation of gut microbiome and immunological and inflammatory markers which are individual in each patient, as illustrated in FIGS. 6 and 7.

The assembling specialty team may be chosen based on synergistic knowledge capabilities in multiorgan pathophysiological processes occurring in chronic conditions. The specialties were chosen after performing research and detailed analysis in each specialty scope of practice expertise and available certified post graduate targeted training. The specialists were chosen to be able to evaluate patients as a complex meta-organism, intercommunicating system of trillions of bacterial cells that coexist with a roughly equal number of human cells in the body.

Each new clinic will have these five (5) specialists as a golden standard for physician specialist model and can be altered after two years data analysis of practice outcome or in the face of new discoveries in pathophysiology or treatments of chronic conditions.

Therefore, the clinics embodied in the present invention is not like existing multispecialty group that are assembled based on prevalence and distribution of most common conditions. Our specialists were assembled to better explore, evaluate and treat not just symptoms but multiple processes involved in chronic condition like immune responses, inflammations, and not clinically evident infections.

The plurality of specialists will practice medicine in different way than existing multispecialty groups. These multi-specialty practices depend on referrals from primary care physician and deliver single specialty consultation pertinent to their specialty. As defined herein, the plurality of specialists will work in a collaborative way, where the results and determinations for each patient will be reviewed and seen by all specialist. Therefore, we will be delivering single, more comprehensive plan of care based on the plurality of at least five (5) physician specialist collaborative recommendations. This assigning and matching of plurality of physician specialists expands significantly beyond previously publish approach of predicting cost and project resources assignment on the large-scale health delivering organizations or insurance company, as taught in the prior art. For instance, the score of 100 does not tell ER providers if cardiologist, or nephrologist should be called. The score does not guarantee of seeing multiple specialist in the emergency room. In our model, the patient will see all specialist on each visit and received multidisciplinary one integrated plan of care. The patient will not need additional referrals, and the patient will have easy guaranteed access to specialist (notably, some specialists do not take particular insurance and are not allowed to deliver care to Medicare and Medicaid members, the population with highest prevalence of multiple chronic conditions). None of the prior art addressed or was able to overcome this existing large barrier in healthcare delivery. The first visit of embodied in the present invention can substitute for a minimum of five visit, that traditionally patients with multiple chronic conditions would have to make over five separate visits to discuss each of the specialist recommendations and update primary care records. The savings in the direct cost of care delivery and associated costs will be calculated and monitored in our solutions.

### Function of Laboratory Equipment

Clinics will have predefined laboratory equipment which will allow very specific testing. The assigned combination of tests will be used as a standard evaluation in each patient; before or during the systemic first visit. Although each single test is use in different specialist offices the assembled combination of these tests—“battery of tests”—is not used in primary care or even specialist office as a standard. The combination of tests of the present invention have been arranged with the purpose of in-depth analysis of pathophysiological processes involved in patients with chronic conditions and relates to new pertinent research and discoveries.

The standard testing for each patient in the clinic will include in addition to traditional laboratory testing following studies: blood levels of inflammatory markers like CRP, erythrocyte sedimentation rate, acute phase proteins and immune activation markers like immunoglobulin classes and levels, levels of neopterin, and cytokines and comprehensive stool studies to evaluate gut microbiome. The extent of stool testing are part and parcel of the comprehensive evaluation due to new fast expanding research in this area. Evaluation may include multiple tests to evaluate microbiota of the gut. The clinic will have capability of preserving stool samples for further research or additional studies.

What we propose as a novel approach is very targeted equipment that is used not for some patients as needed (like ECG testing or X-rays) but equipment that is defined as standard lab for all patients as part of more comprehensive functional evaluation of patients with chronic conditions. Our testing equipment was chosen to correlate with the fields of expertise of practicing physicians.

At the present time none of the primary care doctors evaluate patients stool as standard of care. None of the specialist evaluate stool unless patient has diarrhea or inflammatory bowel diseases. The studies are made impossible due to the insurance approval for diagnostic studies based on CPT codes diagnosis. Therefore, assigning to clinic specific laboratory equipment when combine with a different way of practice will allow to propose unique and value-based care. The practice will allow to perform proposed studies independent of patient insurance or economic status. The practice can dramatically improve care among most needy populations. Therefore, we can show that standards of testing and model of care practice in primary care clinics are significantly changed and expanded.

Providing a Signature Medical History Describing a Patient from Said Pre-Existing Clinic, Said Patient Having at Least One Chronic Condition

The developing of signature medical records for novel model of practice cannot be compared to standard procedures implemented through electronic EMR as in the prior art and cannot be described as mere automatization and simplifying method of patient chart creation, maintenance and retrieval.

All existing commercial EMR have predefined sections pertinent to patient diagnosis, laboratory findings, vitals recording, metrics of quality like HEIDIS, claims, insurance and referrals. At the present time you are buying predefined EMR.

In addition to standard sections there is need customization of fields and correlated functions. Since there is no historical data regarding multidisciplinary practice and collaborative decision making, the present invention aims to develop processes of collaborations between specialists and programmers to develop correct assigning of decision making data fields not used in todays practice—like severity, presence or absence of inflammatory responses, severity, presence or absence of immune activation, number of organs affected, numbers of system in the body involved, importance of finding scored by single vs collaborative scoring of the five (5) physician specialist, percentage of decision made unanimously as opposed to a by majority vote are a few examples of need for a novel monitoring of practice approach not done in primary care previously. In addition, EMR records should provide data that shows signature microbiome of each patient based on their results and stool studies. The established “fingerprint microbiome” should be evaluate and compare overtime with analysis showing trends and deviations over time or in response to treatment. This type of customization that will be required cannot be called an automatization and simplifying method of patient chart creation, maintenance and retrieval; rather, it involves utilization of high level knowledge and creation new solutions that will require new software programming done as collaboration between physicians familiar with particular brand of EMR and programmers.

The data in regard to signature microbiome may be adapted to connect to clinical data to evaluate the importance of each component in the clinical picture since it is unknown which microbe is an accidental finding or is related to certain condition as marker, or it is a culprit causing the conditions. Each patient is serving as their own control over the time and results may be adapted to relate to particular geographical area and include distal patients travel.

Calculating a Risk Score Related to Primary, Secondary, and Tertiary Care Associated with the at Least One Chronic Condition Based in Part on the Signature Medical Record.

There are many scoring systems used in medicine. The present invention calls for developing a new unique scoring system for patients with chronic conditions that can serve not only as a risk score but serve as a score used for treatment of patient and take into account complex processes of immune modulations in human body, inflammation, infections and presence of “fingerprint” of gut microbiome. The scoring system proposed by the present invention differs significantly from the prior art.

Analyzing new markers will require a first evaluation of the impact of each of the factors (bacteria, virus, phage on the individual stage of disease or symptoms) since there is no historical data containing this information. Therefore, processes not coded by ICD—9 or 10—have to be entered to modes, and weight of each new finding has to be assigned by specialist. Thus, pre-existing score calculating modem is not useful to everyday practice in the new outpatient clinic. The data input and variables must be expanded and weighted by significance of altering the disease condition after specific predefined time of monitoring. Likewise, the present invention differs significantly from scoring system developed by Ong (prior art), which developed scoring modules for calculating the risks score based on a binary prediction of a plurality of weight classifiers which are based on past data provided by data access modules. The limitation of binary prediction lays in the assumption that middle value is the median of all the values. The program cannot be applied to multiple interaction between complex biological processes, which the present invention engages in. The prior art process was used to evaluate risk for a one event—heart attack and all variables used for the evaluation were established known factors with known distribution.

In other words, the binary coding of answers yes or no does not reflect complex multiple conditions and multiple variables relationships. The present invention process of developing risk scores system is adapted to utilize a kind of analysis where an answer can reflect not only presence or absence of a factor, but can test causative relationship, for example bacteria X can appear in certain diseases but is just associated or detected and not causing any symptoms when bacteria Y is associate with disease can cause the symptoms and probably or for sure is the cause of the disease.

The scoring system of the present invention may be used not for prediction of one acute event but for evaluating of impact and relationship to many diseases and symptoms. The score will serve to evaluate the need of immune modulating therapies and monitoring outcomes.

The inventive risk score concept is different also than described by Eisenberg who develop system to predict Down syndrome in the newborn based of maternal levels of alpha-fetoprotein (MSAFP) and demographic data. The score predicts a single disease using AFP results that can be the graphed normalized to spam range of Multiples of Median (MoM) range based on normal range of healthy population. The MoM and normal range of human microbiome connected to geographical area of single practice of few thousand, with a presence of single or multiple chronic conditions is unknown. Therefore, Eisenberg method of establishing risk score based on single critical laboratory value cannot be utilized for estimation of impact of multiple laboratory values with unknown normal range distribution.

As described herein our risk score was not design to assess risk for a single acute illness, event or disease. The present invention employs a new unique scoring system for patients with chronic conditions that can serves to guide treatment of patient and consider complex processes of immune modulations in human body, inflammation, infections and presence of “fingerprint” of gut biome in the art of practicing medicine. The present invention is developed to deliver care to patients with chronic conditions, a complex disease that at the present time for thousands of affected people remains uncurable.

The growing epidemic of chronic diseases, lack of cures and staggering cost of health care for these patients proves that there is nothing obvious in the art of assessing risk in chronic conditions. The assessing risk for a disease that has unknown origins (majority chronic conditions) or are not well establish pathophysiology and does not have a cure requires more than analysis of potential expenses or matching specialist based on prevalence and geographical area. (MC CArthy) The risk of progression, the potential of regression and cure, the correlations between multiple chronic conditions need a further study and unconventional solutions/clinics that these patients can turn for help.

### Formulating a Plan of Care for the Patient Based on the Signature Medical Record and on the Plurality of Physician Specialists, Wherein the Plan of Care is Formulated Before a First Visit.

As we disclosed herein, the plurality of specialist will work in a collaborative way, and each patient and his results will be reviewed and seen by all specialist. Therefore, we will be delivering single, more comprehensive plan of care including the at least five (5) specialist collaborative recommendations. The role of the primary care physician as a gate keeper and coordinator is eliminated in this model. Therefore, the process is not similar to the prior art.

Providing a Collaborative Consultation Between the Patient and Each of the Plurality of Physician Specialists within a Predetermined Time Frame of the First Visit, Thereby Eliminating a Primary Physician Referral of the Plurality of Physician Specialists.

The process of care delivery provides primary secondary and tertiary care without need of primary care physician of coordination, delivering more comprehensive and expanded care to the inherently complex chronic-condition patient, eliminating many barriers existing in the present care delivery model.

The elimination of role of primary care physician as care coordinator, gate keeper underlines the significance of change of care delivery for patients with chronic conditions. The establishment of novel clinic is not like any electronic inventions intended to improve referrals or to deliver faster care in emergency room. The present invention proposes an improvement in efficiency of referrals by computer program that can automatically schedule appointments based on uploaded schedules of many specialist calendars. We propose to eliminate this referral system. The clinic of the present invention does not have to refer patients to specialist (our clinic will refer a patient if needed to the hospital or for invasive procedures). The present invention will deliver service provided by primary care physicians and specialist at the same place at the same visit for majority of needed everyday outpatient care.

The advanced experts specialist working in collaborative and synergistic model will evaluate patient additionally in non-traditional way, looking at the human body as a “meta-organism”, a complex system communicating between trillions of bacterial cells and trillions of human cells in the body.

Providing as a standard of care incorporating non-invasive testing or procedures refers to a description of “set up” in primary care, including procedures like cardiac catheterizations, endoscopies, vascular interventions. The definition of non-invasive procedure meant to describe “set up” or scope of practice of standard primary care office in order to differentiate our practice from existing multi-specialty physician groups, where specialist perform advanced invasive procedures and studies. The laboratory studies in the practice will involve I combination of standard blood draws, venipunctures and less invasive sample accumulation as needed and allowed by provided equipment.

The clinic is designed to have ability to implement therapy based on an immune and bio-modulating factors such as: well-known prebiotics, probiotics or symbiotics, antibiotics and biological stimulators—like for example special diet. These therapies can be added to standard therapies based on accumulated data—including new approach of microbiome evaluation from stool samples and with provided expertise of multi specialist team and with provided expertise of specialist team. The immune modulating therapies may involve following steps:
evaluating and determining patient immune status
obtaining biological samples (stool and blood)
performing a blood assay and stool studies
determining the patient's gut microbiome (microbiota living in human gastrointestinal tract)
developing and validating a scoring system for evaluation of severity and extend of diseases based on new listed before immune and pathophysiological markers
calculating patient risk score
administering of specific type immune modulating therapies
monitoring outcomes

The probiotics are well known over 100 years and include Lactobacillus and Bifidobacterium, however are not at standard use at the primary care clinic for patients with chronic conditions.

The prebiotics were established in 2007 and describes as selectively fermented ingredient that allows specific changes, both in the composition of and/or activity in the gastrointestinal microflora that confers benefits upon a host's well-being and health. This definition means that the ingredients of prebiotics should not be metabolized by a human host's cells and may only be metabolized by members of the gut microbiota considered to be important to gut health, such as the lactobacilli and bifidobacterial. At the present time only two food ingredients, insulin and galactooligosaccharides (GOSs), fulfill these criteria.

The treatment in primary care setting can utilized an administration of combination of prebiotics and probiotics since they are known to have synergistic effect for restoring gut microbiome to healthy state. The probiotics given together with prebiotics are called symbiotics. The immune modulating treatment can involve also immune stimulation with specific diets. These compounds are easily available non-prescription items. However, the impact of each compound and outcomes on chronic conditions are not known. This is a very fast-growing field that scientist are looking into new DNA techniques and new treatments built into the new model of care delivery that potentially can implement all these discoveries in the future but at the same time can deliver so needed expanded care to complex patients with chronic conditions. The data and samples collected in the new model enable better insight to pathophysiology of many diseases.

The computer-based data processing system and method described above is for purposes of example only and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the present invention.

What is claimed is:

1. A treatment method comprising:
determining a fingerprint of gut microbiome measured in a biological sample from a patient diagnosed with a chronic condition to identify the patient having at least one immunological marker and at least one inflammatory marker;
administering a treatment to the patient having an immunological marker and an inflammatory marker of the chronic condition, wherein the treatment is a course of immune modulation therapy capable of treating the chronic condition,
wherein the course of immune modulation therapy comprises administering prebiotics, probiotics, symbiotics, antibiotics or biological stimulators, wherein the course of immune modulation therapy comprises an assessment from an infectious disease specialist based on the at least one immunological marker in view of the fingerprint of gut microbiome.

2. The method of claim 1, wherein the course of immune modulation therapy comprises an assessment from a cardiologist based on the at least one inflammatory markers of the patient in view of the fingerprint of gut microbiome.

3. The method of claim 2, wherein the course of immune modulation therapy comprises an assessment from a gastroenterologist based on an immune status of the patient in view of the fingerprint of gut microbiome.

4. The method of claim 3, wherein the course of immune modulation therapy comprises an assessment from a gastroenterologist based on a gastrointestinal tract of the patient in view of the fingerprint of gut microbiome.

5. The method of claim 4, wherein the course of immune modulation therapy comprises an assessment from an endocrinologist based on or more pathophysiological markers of the patient in view of the fingerprint of gut microbiome.

6. The method of claim 5, wherein said assessments consider a signature medical history of the patient.

7. The method of claim 6, further comprising calculating a risk score related to primary, secondary, and tertiary preventive care associated with the patient based in part on the signature medical record.

8. The method of claim 7, further comprising formulating a plan of care for the patient based on the signature medical record and on a plurality of said specialists, wherein the plan of care is formulated before a first visit with the patient.

9. The method of claim 8, further comprising providing a collaborative consultation between the patient and each of the plurality of said specialists within a predetermined time frame of the first visit, thereby eliminating a primary physician referral of the plurality of said specialists.

10. The method of claim 9, wherein the fingerprint of gut microbiome is the genetic signature of gut microbiota' refers to the gastrointestinal tract of the patient.

11. The method of claim 10, wherein the fingerprint of gut microbiome is the determined through stool studies of the patient.

12. The method of claim 11, wherein the plurality of assessments is based in part on a blood levels of inflammatory markers, an erythrocyte sedimentation rate, and acute phase proteins.
